(19) United States
(12) Patent Application Publication
(10) Pub. No.: US 2020/0126395 A1
(43) Pub. Date: Apr. 23, 2020

(54) HYGIENE MONITORING SYSTEM AND METHOD

(71) Applicant: Sanus Solutions, LLC, Atlanta, GA (US)

(72) Inventors: Luka Antolic-Soban, Atlanta, GA (US); Jianming Zeng, Atlanta, GA (US); Junxian Wu, Atlanta, GA (US); Hongrui Zheng, Atlanta, GA (US)

(21) Appl. No.: 16/556,453

(22) Filed: Aug. 30, 2019

Related U.S. Application Data
(60) Provisional application No. 62/725,254, filed on Aug. 30, 2018.

(51) Int. Cl. G08B 21/24; A61B 5/00

(52) U.S. Cl. CPC G08B 21/245; A61B 5/002

(57) ABSTRACT

A system and method of monitoring the hand washing protocol of a person in contact with a medical patient. The system includes a camera which detects the presence of a person entering the room and takes a photograph of that person. The system also includes a first hand hygiene device and a first sensor which detects the use of the first hand hygiene device. The hygiene monitoring system also includes a second hand hygiene device and a second sensor. The camera, first sensor, and second sensor are in communication with a local data computer or computer server through an Ethernet port or conventional wireless router. The system takes a photograph of the hospital employee entering the room, identifies that person through facial recognition software, and determines if that person has followed proper hand washing protocol by monitoring the sensors associated with that room.

### REFERENCE TO RELATED APPLICATION

Applicant claims the benefit of U.S. Provisional Patent Application Serial No. 62/725,254 filed Aug. 30, 2018 and entitled HUMAN HYGIENE MONITORING ASSISTANT.

### TECHNICAL FIELD

This invention relates generally to a system and method for monitoring hygiene protocols, and more particularly to a system for monitoring and recording the hand washing of people.

### BACKGROUND OF INVENTION

In today's working environment, it is oftentimes required that employees follow strict specific work practices to avoid hazards or the creation of an unsafe result. However, employees may be less knowledgeable, forgetful, or less experienced in the protocol associated with their work. These problems may have a negative impact on their employee, work performance, or services provided by the employee.

A prime example of following workplace protocol involves the simple task of medical personnel washing their hands properly prior to coming in contact with the patient. This task has proven to be difficult to achieve on a regular basis, yet may have dire consequences if not completed. In the United States alone, hand hygiene compliance rates in an intensive care unit may be as low as approximately 33%, even though the results of not doing such may be the patient contracting an infection that may result in death.

Systems have been developed to monitor the hand washing of medical personnel. For example, U.S. Patent Application Serial No. 2016/0267772 A1 shows a tracking system for hand hygiene. This system uses an electronic badge which has wireless communication capabilities. The badge communicates with a hub which tracks the location of the badge to determine compliance with the hygiene protocol. A problem with this type of system is that people sometimes forget to wear the badge, resulting in the person not being properly tracked for compliance purposes. Another problem with these types of systems is that the decision of whether the person washed their hands or not is determined by physical location, wherein the physical location does not necessarily confirm that the hand washing actually took place.

Accordingly, it is seen that a need remains for a system for monitoring the hand washing hygiene of medical personnel. It is to the provision of such therefore that the present invention is primarily directed.

### SUMMARY OF THE INVENTION

A hygiene monitoring system for monitoring employees within a patient room having a doorway comprises a computer having facial recognition software for producing real time facial embeddings from a captured image, a database of previously stored employee facial embeddings, facial comparison capabilities to compare real time facial embeddings to the database of previously stored employee facial embeddings, a first hand hygiene device, a first hand hygiene device sensor coupled to the first hand hygiene device and electronically coupled to the computer, and a camera electronically coupled to the computer and positioned within the room to capture an image of an employee entering the room through the doorway and transmitting the captured image to the computer for facial comparison by the computer. With this construction, the computer determines if the employee has actuated the first hand hygiene device prior to interacting with a patient within the patient room.

A method of monitoring hygiene of an employee entering a patient room through a doorway comprises the steps of providing a computer having facial recognition software, a first hand hygiene device, a first hand hygiene device sensor, and a camera, capturing an employee image of an employee as that employee enters the patient room, determining the identity of the employee through the employee image and the facial recognition software, determining if that employee has utilized the first hand hygiene device through actuation of the first hand hygiene device sensor, and initiating an alert signal if it is determined that the employee has not utilized the first hand hygiene device.

### BRIEF DESCRIPTION OF DRAWINGS

FIG. 1 is a schematic view of a hygiene monitoring system embodying principles of the invention in a preferred form.

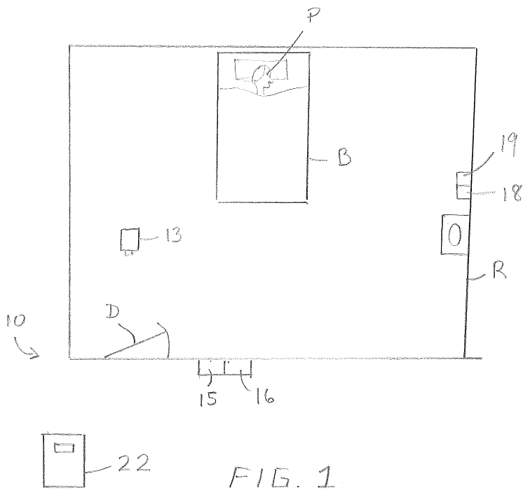

FIG. 2 is a schematic view of the system architecture of the hygiene monitoring system of FIG. 1.

FIG. 3 is a flow chart showing the use of the hygiene monitoring system of FIG. 1.

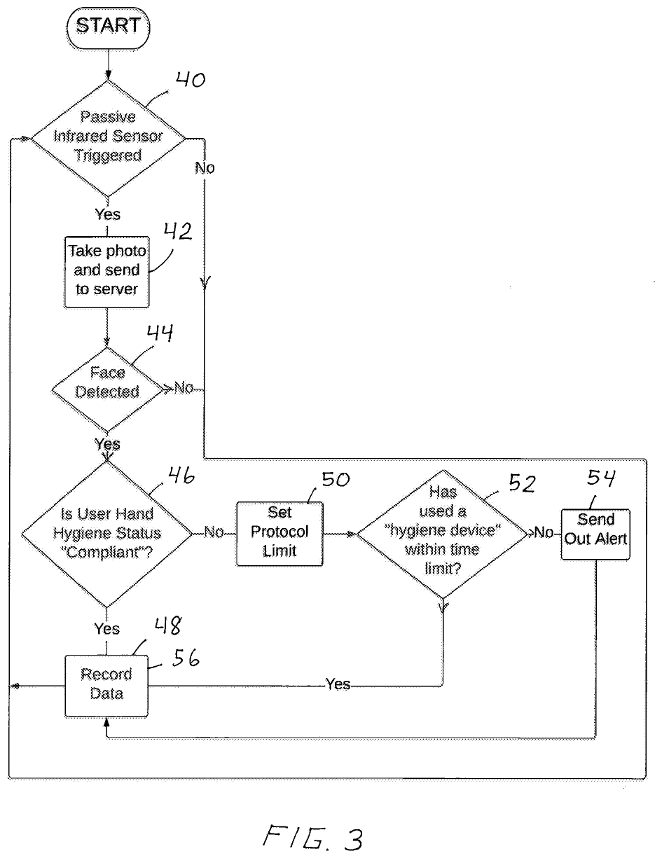

### DETAILED DESCRIPTION

With reference next to the drawings, there is shown a hygiene monitoring system 10 of the present invention in a preferred form. The hygiene monitoring system 10 includes an Internet of Things (IoT) network of embedded devices which is designed to work in a patient care facility having multiple patient rooms R. Each room R has at least one patient bed B upon which is positioned a patient P.

Specifically, the hygiene monitoring system 10 includes a wireless transmitting camera microcomputer unit 13 (referenced hereinafter simply as “camera”) which is mounted to the ceiling within a patient's room R. The camera 13 is positioned to detect the presence of a person entering the room R through a doorway or door D and subsequently to take a photographic image of that person. The camera 13 may be, for exemplary purposes only, a Raspberry pi 3 B+ microcomputer having a passive infrared sensor, Raspberry Pi Camera Module V2-8 Megapixel, 1080P, and output devices of a mini external USB stereo speaker and LED light.

The hygiene monitoring system 10 also includes a first hand hygiene device 15 mounted to the exterior surface of room wall adjacent the room door D to distribute a hand sanitizer in conventional fashion, such as through a washing sink or hand solution sanitizer. The first hand hygiene device 15 includes or is mounted closely adjacent a wireless first hand hygiene device attachment unit or sensor 16. The first sensor 16 detects the use or actuation of the first hand hygiene device 15 through any conventional means, such as through the motion detection of a person or the physical activation of the first hand hygiene device 15 through an electronic or mechanical switch. The first sensor 16 includes a camera component so that it may take a photographic image of the person utilizing the first hand hygiene device 15. The first hand hygiene device 15 and sensor 16 may be designed as two separate units or as a single or unitary device.

The wireless first hand hygiene device sensor 16 may be, for exemplary purposes only, an infrared sensor such as a Raspberry pi 3 B+ microcomputer having a 5V Pololu Carrier with Sharp Gp2YOA60Sz Ir Range Sensor 10 to 150 cm with a Raspberry Pi Camera module V2-8 Megapixel, 1080p, and output devices of a mini external USB stereo speaker and LED light.

The hygiene monitoring system 10 also includes a second hand hygiene device 18 mounted to the interior surface of room wall to distribute a hand sanitizer in conventional fashion, such as through a washing sink or hand solution sanitizer. The second hand hygiene device 18 includes or is mounted closely adjacent a wireless second hand hygiene device attachment unit or sensor 19. The second sensor 19 detects the use or actuation of the second hand hygiene device 18 through any conventional means, such as through the motion detection of a person or the physical activation of the first hand hygiene device through an electronic or mechanical switch. The second sensor 19 includes a camera component so that it may take a photographic image of the person utilizing the second hand hygiene device 18. Again, the second hand hygiene device 18 and sensor 19 may be designed as two separate units or as a single or unitary device.

The wireless second hand hygiene device sensor 19 may be, for exemplary purposes only, an infrared sensor such as a Raspberry pi 3 B+ microcomputer having a 5V Pololu Carrier with Sharp Gp2YOA60Sz Ir Range Sensor 10 to 150 cm with a Raspberry Pi Camera module V2-8 Megapixel, 1080p, and output devices of a mini external USB stereo speaker and LED light.

The first and second hand hygiene device sensors 16 and 19 are coupled to their respective first and second hand hygiene devices 15 and 18, the term coupled meaning that the sensors sense the presence of a person at the physical location of the devices 15 or 18 or senses the physical actuation of the devices 15 or 18, through motion sensing, electronic switches, mechanical switches, or other similar means. As such, the sensors may simply be positioned to sense without being physically or mechanically coupled to the hand hygiene devices 15 and 18.

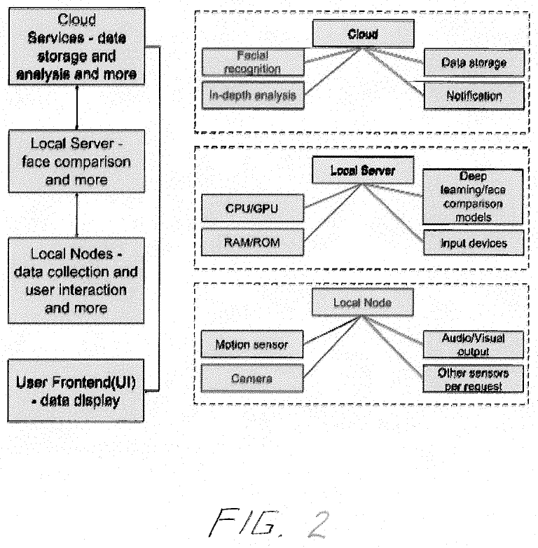

The camera 13, first hand hygiene device sensor 16, and second hand hygiene device sensor 19 are preferably in wireless electronic communication with a local data computer or computer server 22 through an Ethernet port or conventional wireless router, such as a Netgear Nighthawk Smart WiFi Router (R6700) made by Netgear, Inc. of San Jose, Calif., which establishes a local area network (LAN) that is connected to the internet for real time information processing. The data server 22 hosts conventional data streaming service, a computer vision service, a web framework, and a local database, with the potential of migrating all services to an offsite or cloud location and therefore such cloud related actions are considered to be on the server 22. The server 22 or system may include, for example, data streaming such as Apache Druid made by Apache Software Foundation, a web application such as Apache superset which enables information or data regarding specific people, rooms, departments, units, area traffic flow, real time location of staff, compliance rates for specific staff members, departments, units, or overall hospitals, report generating capabilities, and the ability to access, query, and export data for other analysis purposes through Excel and Tableau software. The server 22 also includes computer vision/machine learning software such as Tensorflow, web server software such as Flask with GUnicorn, local database software such as Mongo DB which may provide specific times, names, dates, rooms for complaint and non-compliant events, a cloud database software such as Amazon Web Service (AWS), cloud facial recognition software such as Amazon Web Service (AWS) Rekognition, cloud messaging service software such as Amazon Web Service (AWS) simple notification service, and a docker which uses operating-system-level virtualization to develop and deliver software in packages called containers. This system architecture is shown in FIG. 2, wherein the cloud service component includes the facial recognition, in-depth analysis, data storage and notification for the system. The local server 22 includes the CPU or GPU, memory (RAM/ROM), facial comparison, and input devices (camera and sensors). The local node includes the actual components in the patient room, such as the sensors, camera, audio/visual output, or other sensors which monitor actions within the room.

In use, each health facility staff member or personnel that needs to be monitored for hygiene compliance must provide a facial photograph or image which is entered into and stored within the system 10, specifically the server 22. The photograph may be taken for this distinct purpose or may be provided from an identification badge associated with that person.

The photograph is sent to and stored at the local data server 22 along with other personal data identifying information such as the person's name, title/position, age, etc. The server 22 converts the photograph into an embedding or face or facial embedding, which is a group of numerical vectors that represent each detected face in the photograph in a 1024-dimensional space. The embedding is stored along with the employee's personal data so that the employee may now be identified and monitored by the system 10.

As the health facility employee enters a patient's room R the ceiling camera 13 captures a facial image of the employee, as shown by step 42. This facial image is then sent to the server 22 where it is converted to a facial embedding, where the facial embedding is analyzed and compared to the database of employee facial embeddings stored within the server 22, shown as step 44. Once an employee comparison is successfully found through facial recognition, the server 22 compares the employee to his or her hygiene status data saved on the local data server 22.

Should the employee have just washed his or her hands at the first hand hygiene device 15 just outside the door D of the patient's room R, shown as step 40, this hand washing event will have been sensed by the first hand hygiene device sensor 16 and transmitted to the server 22 for recording. The first sensor 16 will have captured a facial image of the employee utilizing the first hand hygiene device 15, sent the captured image to the server 22 for conversion to a facial embedding and comparison to the database to identify the employee. As such, when the server 22 compares the employee to his or her hygiene status data, the results are the proper hand washing protocol had been followed and notification of improper protocol is not generated, shown as step 46, i.e., the system establishes that the employee captured by room camera 13 is the same employee that used the first hand hygiene device 15 outside the room, thus establishing a proper hand washing protocol. The system records the data related to the employee on the server 22 for proper recording keeping, as shown as step 48.

However, if the employee had not washed his or her hands at the first hand hygiene device 15, the server's negative comparison of the captured image through camera 13 and through first sensor 16 will result in a potential breach of the hand washing protocol. The potential breach causes the server 22 to commence a preselected time interval or limit to comply with the hand washing protocol, for example, a 30 second time limit, as shown as step 50.

If after the preselected time limit, as shown as step 52, the employee has not complied with the protocol by washing his or her hands, as established through the use of either the first or second hand hygiene device and the captured image through their respective sensors 16 or 19, the system records the breach and initiates a real-time alert, as shown as step 54. This completes the system's activation protocol. However, the system may continue to send alerts to the employee until a hand washing event has been detected by a sensor 16 or 19. The alert may be an audio cue through a speaker attached to the camera 13, through a text message, sound alert, or tactic alert to a portable device or phone carried by the employee.

If on the other hand the employee has washed his or her hands, which is indicated by the activation of either the first sensor 16 at the first hand hygiene device 15 or the second sensor 19 at the second hand hygiene device 18 and their respective successful facial recognition image comparison through sensors 16 or 19, then the system records the data, as shown as step 56.

The recorded data may include the employee information, room number or location, hygiene status, time, patient information, or other pertinent data related to the event.

The system 10 may use the software to generate reports, graphs and trends concerning compliance rates for specific staff members, departments, units or overall hospitals.

It should be understood that although the system has been described with particular reference to a hospital environment, the present invention may also be adapted for other industries, such as in the food preparation industry wherein the washing of a person's hands is important prior to the employee preparing food.

It should also be understood that should multiple employees enter the room at nearly the same time, the system will analyze each employee for protocol compliance, through the facial recognition of each at either the first or second hand hygiene devices 15 or 18.

It thus is seen that a method and system for utilizing facial recognition to monitor and report hygiene protocol compliance is now provided which overcomes problems associated with the prior art. While this invention has been described in detail with particular references to the preferred embodiments thereof, it should be understood that many modifications, additions and deletions, in addition to those expressly recited, may be made thereto without departure from the spirit and scope of the invention.

1. A hygiene monitoring system for monitoring employees within a patient room having a doorway, the hygiene monitoring system comprising:
a computer having facial recognition software for producing real time facial embeddings from a captured image of an employee, a database of previously stored employee facial embeddings, facial comparison capabilities to compare said real time facial embeddings to said database of previously stored employee facial embeddings;
a first hand hygiene device;
a first hand hygiene device sensor coupled to said first hand hygiene device and electronically coupled to said computer, and
a camera electronically coupled to said computer and positioned within the room to capture an image of an employee entering the room through the doorway and transmitting the captured image to said computer for facial comparison by said computer,
whereby the computer determines if the employee has actuated the first hand hygiene device prior to interacting with a patient within the patient room.

2. The hygiene monitoring system of claim 1 wherein said first hand hygiene device sensor includes a camera positioned to capture the image of an employee utilizing said first hand hygiene device.

3. The hygiene monitoring system of claim 1 wherein said computer also includes a preselected timer, and wherein said computer initiates an electronic alert signal if said first hand hygiene device sensor is not activated prior to or within the expiration of said preselected timer from said camera capturing the image of the employee.

4. The hygiene monitoring system of claim 1 wherein said first hand hygiene device and said first hand hygiene device sensor are mounted outside the patient room adjacent the doorway.

5. The hygiene monitoring system of claim 1 wherein said first hand hygiene device and said first hand hygiene device sensor are mounted inside the patient room.

6. The hygiene monitoring system of claim 4 further comprising a second hand hygiene device and a second hand hygiene device sensor coupled to said second hand hygiene device and electronically coupled to said computer, and wherein said second hand hygiene device and said second hand hygiene device sensor are mounted inside the patient room.

7. The hygiene monitoring system of claim 6 wherein said second hand hygiene device sensor includes a camera positioned to capture the image of an employee utilizing said second hand hygiene device.

8. A hygiene monitoring system for monitoring employees within a room and a first hand hygiene device associated with the room, the hygiene monitoring system comprising:
a computer having facial recognition software for producing real time facial embeddings, a database of previously stored employee facial embeddings, facial comparison capabilities to compare real time facial embeddings to said database of previously stored employee facial embeddings;
a first hand hygiene device sensor associated with the first hand hygiene device and electronically coupled to said computer,
a camera electronically coupled to said computer and positioned within the room to capture an employee image of an employee entering the room through the doorway and transmitting the employee image to said computer, and
facial recognition software associated with said computer, said facial recognition software being capable of comparing the employee image from said camera to a database of stored employee images to determine the identity of the employee, and determining if the first hand hygiene device sensor has sensed the actuation of the first hand hygiene device, and sending an alert signal if the first hand hygiene device sensor has not sensed the actuation of the first hand hygiene device within a preselected time frame from said hygiene monitoring system sensing the presence of the employee within the room.

9. The hygiene monitoring system of claim 8 wherein said first hand hygiene device sensor includes a camera positioned to capture the image of an employee utilizing the first hand hygiene device.

10. The hygiene monitoring system of claim 8 wherein said hygiene monitoring system sensing the presence of the employee in association with the room through said camera capturing the employee image.

11. The hygiene monitoring system of claim 8 wherein said first hand hygiene device and said first hand hygiene device sensor are mounted outside the room adjacent the doorway.

12. The hygiene monitoring system of claim 8 wherein said first hand hygiene device and said first hand hygiene device sensor are mounted inside the room.

13. The hygiene monitoring system of claim 11 wherein the hygiene monitoring system is used with a second hand hygiene device, and wherein the hygiene monitoring system further comprises a second hand hygiene device sensor coupled to the second hand hygiene device and electronically coupled to said computer, and wherein the second hand hygiene device and said second hand hygiene device sensor are mounted inside the room.

14. The hygiene monitoring system of claim 13 wherein said first hand hygiene device sensor includes a camera positioned to capture the image of an employee utilizing said first hand hygiene device.

15. A method of monitoring hygiene of an employee entering a patient room through a doorway, the method comprising the steps of:
(A) providing a computer having facial recognition software, a first hand hygiene device, a first hand hygiene device sensor, and a camera;
(B) capturing an employee image of an employee as that employee enters the patient room;
(C) determining the identity of the employee through the employee image and the facial recognition software;
(D) determining if that employee has utilized the first hand hygiene device through actuation of the first hand hygiene device sensor, and
(E) initiating an alert signal if it is determined that the employee has not utilized the first hand hygiene device.

16. The method of claim 15 wherein step (D) the determination is made by capturing a second photographic image of the employee utilizing the first hand hygiene device and identifying the employee through the facial recognition software.

17. The method of claim 16 wherein step (A) the computer also includes a preselected timer, and wherein the computer initiates an electronic alert signal if the first hand hygiene device sensor is not activated within the expiration of the preselected timer from the camera sensing the employee.

18. The method of claim 16 wherein the first hand hygiene device and first hand hygiene device sensor are mounted outside the patient room adjacent the doorway.

19. The method of claim 16 wherein the first hand hygiene device and the first hand hygiene device sensor are mounted inside the patient room.

20. The method of claim 18 wherein step (A) a second hand hygiene device and a second hand hygiene device sensor coupled to the second hand hygiene device and electronically coupled to the computer are provided, and wherein the second hand hygiene device and the second hand hygiene device sensor are mounted inside the patient room.

21. The method of claim 20 wherein a determination is made by capturing a second photographic image of the employee utilizing the second hand hygiene device and identifying the employee through the facial recognition software.
